(19) United States
(12) Patent Application Publication
(10) Pub. No.: US 2014/0296890 A1
(43) Pub. Date: Oct. 2, 2014

(54) Secondary pigmentary glaucoma iris scraping treatment method and iris scraping tool

(71) Applicants: Eva M. T. Slaughter, Buffalo, NY (US); Tawan S. T. Slaughter, Buffalo, NY (US); Javon Z. T. Slaughter, Buffalo, NY (US)

(72) Inventors: Eva M. T. Slaughter, Buffalo, NY (US); Tawan S. T. Slaughter, Buffalo, NY (US); Javon Z. T. Slaughter, Buffalo, NY (US)

(21) Appl. No.: 14/120,464

(22) Filed: May 21, 2014

Related U.S. Application Data
(63) Continuation-in-part of application No. 13/068,238, filed on May 6, 2011, now abandoned.
Application No. 13/068,238 is a continuation-in-part of application No. 12/347,941, filed on Dec. 31, 2008, now abandoned.

(51) Int. Cl. A61B 17/32

(52) U.S. Cl. CPC A61B 17/32; A61B 2017/320008
USPC 606/161

(57) ABSTRACT

An iris scraping surgical method using an iris scraping tool treats secondary pigmentary glaucoma. The iris scraping tool is inserted through an incision in the sclera made with a separate surgical instrument. The iris is lifted away from the lens and pigmentary debris and cellular debris gently scraped from the backside of the iris to relieve pressure buildup caused by the debris blocking fluid flow between the iris and the lens. An iris scraping convexly curved dull edge of the iris scraping blade conforms to the underside of the lifted iris. A lens clearing concavely curved dull edge conforms to the shape of the adjacent lens surface.

### CROSS-REFERENCE TO RELATED APPLICATIONS

The present utility patent application is a continuation-in-part application of applicant's utility patent application No. 13/068,238 filed May 6, 2011 and applicant's utility patent application No. 12/347,941 filed Dec. 31, 2008 and further claims the benefit of applicant's utility patent No. 7,854,741 issued Dec. 21, 2010.

### STATEMENT REGARDING FEDERALLY SPONSORED RESEARCH OR DEVELOPMENT

Not Applicable.

### THE NAMES OF THE PARTIES TO A JOINT RESEARCH OR DEVELOPMENT

Not Applicable.

### BACKGROUND OF THE INVENTION

1. Field of the Invention

The present invention relates to secondary pigmentary glaucoma treatments and particularly to a surgical iris scraping treatment and iris scraping tool for secondary pigmentary glaucoma, wherein excess pigment debris and/or cellular debris blocks passage of fluid between the iris and the lens, and wherein an incision is made in the sclera with a separate cutting tool to admit the non-cutting iris scraping tool, the iris is lifted and a double-curved dull edged and dull point distal end of the iris scraping tool is positioned between the lifted iris and the lens to scrape away accumulated pigment and/or cellular debris which is blocking the passage of fluid between the iris and the lens, from the back pigment epithelium layer on the backside of the iris without damaging the pigment epithelium layer or the lens, with one convexly curved dull-edged scraping surface conforming to the underside of the iris for gently scraping away the excess blocking pigment and/or cellular debris from the underside of the iris without damaging the pigment epithelium layer, and on an opposite edge of the distal end of the iris scraping tool, a concave dull scraping edge conforming to the curvature of the lens of the eye for gently removing any pigment and/or cellular debris from off of the lens without damaging the lens, while the dull distal end point between the curved scraping edges of the iris scraping tool does not contact the iris or the lens; wherein removal of the blocking pigment and/or blocking cellular material permits the normal fluid flow restored to a normal rate from the vitreous humor and posterior chamber of the aqueous humor out between the iris and the lens to the anterior chamber of the aqueous humor; and wherein the distal end of the iris scraping tool having two opposing dull edged curved scraping surfaces and the handle are preferably fabricated of aluminum coated with a chemical and heat resistant plastic.

2. Description of Related Art including information disclosed under 37 CFR 1.97 and 1.98

Worldwide, glaucoma is the leading cause of irreversible blindness. In fact, as many as six million individuals are blind in both eyes from this disease. In the United States alone, according to one estimate, over three million people have glaucoma. As many as half of the individuals with glaucoma, however, may not know that they have the disease. The reason they are unaware is that glaucoma initially causes no symptoms, and the loss of vision on the side (periphery) is hardly noticeable.

Glaucoma is usually, but not always, associated with elevated pressure in the eye (intraocular pressure). This pressure leads to damage to the eye (optic) nerve. Actually, glaucoma is now considered a disease of the optic nerve (optic neuropathy) that causes a loss of vision, usually in both eyes (bilateral). This loss often begins with a subtle decrease in side (peripheral field) vision. If the glaucoma is not diagnosed and treated, it may progress to loss of central vision and blindness.

Pigmentary glaucoma is a rare form of glaucoma wherein pigment from the iris pigment epithelium is shed thereby releasing pigment particles into the anterior chamber of the aqueous humor which clog the normally draining trabecular meshwork, preventing the drainage of aqueous humor from the anterior chamber.

In certain circumstances, in a type of secondary pigmentary glaucoma, the granules of pigment get stuck on a portion of the bottom layer of the iris between the iris and the lens and partially block the flow of fluid between the iris and the lens to prevent fluid from flowing out of the vitreous humor and posterior chamber of the aqueous humor out into the anterior chamber of the aqueous humor to cause a pressure build up in the eye damaging the optic nerve. This pigmentary glaucoma is a type of secondary glaucoma that is more common in younger men.

Apparently the cause of the pressure build-up in the eye is a build-up of pigment debris and dead cell tissues on the back of the iris which covers the lens of the eye. When the pigment debris and dead cell tissue harden to block the flow of fluid, it causes the level of the pressure in the eye to rise, even to dangerous levels. These levels of high pressure build up in the eye, depend upon the amount of pigment debris and dead cell tissue present.

Iris color is a highly complex phenomenon consisting of the combined effects of texture, pigmentation, fibrous tissue and blood vessels within the iris stroma, which together make up an individual's epigenetic constitution. A person's “eye color” is actually the color of one's iris, the cornea being transparent and. the white sclera entirely outside the area of interest. It is a common misconception that the iris color is entirely due to its melanin pigment; this varies only from brown to black.

Melanin is yellowish-brown to dark brown in the stromal pigment cells, and black in the iris pigment epithelium, which lies in a thin but very opaque layer across the back of the iris. Most human irises also show a condensation of the brownish stromal melanin in the thin anterior border layer, which by its position has an overt influence on the overall color. The degree of dispersion of the melanin, which is in subcellular bundles called melanosomes, has some influence on the observed color, but melanosomes in the iris of man and other vertebrates are not mobile, and the degree of pigment dispersion cannot be reversed. Abnormal clumping of melanosomes does occur in disease and may lead to irreversible changes in iris color (see heterochromia, below). Colors other than brown or black are due to selective reflection and absorption from the other stromal components. Sometimes lipofuscin, a yellow “wear and tear” pigment also enters into the visible eye color, especially in aged or diseased green eyes (but not in healthy green human eyes).

While a number of methods exist for treating pigmentary glaucoma due to the clogging of the trabecular meshwork by pigment debris from the iris, the prior art is lacking in treatments and scraping tools for the secondary pigmentary glaucoma build-up of pigment debris on the back pigment epithelium layer on the backside of the iris blocking the flow of fluid between the iris and the lens.

U.S. patent application Ser. No. 20060241580, published Oct. 26, 2006 by Mittelstein, claims a device and methods useable for treatment of glaucoma, including pigmentary glaucoma, and other surgical procedures. A device and method are provided for cutting or ablating tissue in a human or veterinary patient includes an elongate probe having a distal end, a tissue cutting or ablating apparatus located adjacent within the distal end, and a tissue protector extending from the distal end. The protector generally has a first side and a second side and the tissue cutting or ablating apparatus is located adjacent to the first side thereof. The distal end is structured to be advanceable into tissue or otherwise placed and positioned within the patient's body such that tissue adjacent to the first side of the protector is cut away or ablated by the tissue cutting or ablation apparatus while tissue that is adjacent to the second side of the protector is not substantially damaged by the tissue cutting or ablating apparatus.

U.S. Pat. No. 5,549,596, issued Aug. 27, 1996 to Latina, provides a selective laser method of targeting pigmented ocular cells which involves selectively damaging pigmented cells in an intraocular area by irradiating the area with laser radiation of radiant exposure between about 0.01 and about 5 Joules/cm.sup.2, while sparing non-pigmented cells and collagenous structures within the irradiated area. The method is useful for the treatment of glaucoma, intraocular melanoma, and macular edema.

U.S. Pat. No. 190 6,989,007, issued Jan. 24, 2006 to Shadduck, shows a device and system for non-invasive treatment of a patient's trabecular meshwork to treat primary open-angle glaucoma or pigmentary glaucoma. The system and technique applies energy directly to media within clogged spaces in a patient's trabecular meshwork to increase aqueous outflow facility by (i) localization of microimplantable bodies carrying a selected exogenous chromophore, such as particles with a gold surface, in deeper regions of the trabecular meshwork, and (ii) irradiation of the microimplantables with a selected coherent wavelength having a power, level and pulse duration that is strongly absorbed by the surfaces of the microimplantables.

U.S. Pat. No. 6,682,523, issued Jan. 27, 2004 to Shadduck, claims a system for non-invasive treatment of a patient's trabecular meshwork to treat primary open-angle glaucoma, exfoliation glaucoma and pigmentary glaucoma wherein the meshwork can be clogged with cellular debris and other accumulations. The system and technique applies energy directly to media within clogged spaces in a patient's trabecular meshwork to increase aqueous outflow facility by (i) localization of microimplantable bodies carrying a selected exogenous chromophore in deeper regions of the trabecular meshwork and (ii) irradiation of the microimplantables with a selected coherent wavelength having a power level and pulse duration that is strongly absorbed by the exogenous chromophore. The chromophores are preferably carried in uniform nanocystalline particles having an average diameter ranging from about 0.5 nm to 20 nm. Thermoelastic expansion of the nanoparticles can propagate .+−.10 atm bipolar stress waves in the surrounding fluid media thereby causing microcavitation thereby delivering mechanical energy to ablate debris and accumulations in the meshwork without causing thermal damage to the trabecular meshwork sheets.

U.S. Pat. No. 6,319,274, issued Nov. 20, 2001 to Shadduck, describes an apparatus and technique for transscleral light-mediated biostimulation of the trabecular plates of a patient's eye in a treatment for ocular hypertension or glaucoma, including pigmentary glaucoma. The apparatus includes; (i) a working end geometry for contacting the anterior surface of the sclera and cornea to insure that a laser emission reaches the trabecular meshwork from a particular location on the anterior surface of the sclera, (ii) a laser energy source providing a wavelength appropriate for absorption beneath the anterior scleral surface to the depth of the trabecular plates, and (iii) a dosimetry control system for controlling the exposure of the laser emission at the particular spatial locations. The device uses a light energy source that emits wavelengths in the near-infrared portion of the spectrum, preferably in the range of about 1.30 .mu.m to 1.40 .mu.m or from about 1.55 .mu.m to 1.85 .mu.m. The depth of absorption of such wavelength ranges will extend through most, if not all, of the thickness of the sclera (750 .mu.m to 950 .mu.m). In accordance with a proposed method of trabecular biostimulation, the targeted region is elevated in temperature to a range between about 40.degree. C. to 55.degree. C. for a period of time ranging from about 1 second to 120 seconds or more.

U.S. Pat. No. 4,391,275, issued Jul. 5, 1983 to Fankhauser, discloses a method for the surgical treatment of the eye by perforation, by laser radiation, of a tissue or inner wall of the eyeball having a resistance to the free circulation of the aqueous humour. A laser radiation burst comprising at least one pulse of duration d comprised between 10 and 60 ns and of radiated energy comprised between 30 and 300 millijoules is produced and focused at a determined distance inside the wall. The radiation is focused according to a solid angle .OMEGA. determining a density of radiated energy causing ionization of the propagation medium. A shock wave is also produced due to this ionization close to the mean direction of propagation of the radiated, thereby allowing the tissue or inner wall to be perforated.

U.S. Pat. No. 6,220,247, issued Apr. 24, 2001 to Maldonado Bas, indicates a method of performing trabeculodissection to treat glaucoma using an excimer or galvanometric scanning laser delivery system. A scleral flap is cut to expose the treatment area of the trabecular meshwork. The arc of the treatment area is made as wide as the trabecular meshwork limited by the circumference of the limbal area around the patient's eye. A laser, preferably of the excimer type, is used to treat small test areas in successive discrete zones along the arc of the treatment area in the bed of the scleral flap to determine the precise depth of ablation required over the entirety of each zone to promote filtration without penetration of the treatment zone. The laser then treats discrete zones over the length of the arc to remove in scan layers so as to process discrete ablated zones of minimal residual thickness. The treatment of successive zones allows ablation along the length of the treatment arc without interference from actively draining aqueous. After ablation of the various successive zones, the scleral flap is closed and, if necessary, sutured.

U.S. patent application No. 20050288745, published Dec. 29, 2005 by Andersen, describes a method and device for optical ophthalmic therapy, which includes treatment for different forms of glaucoma, including pigmentary glaucoma. An optical scanning system and method are provided for performing therapy on trabecular meshwork of a patient's eye, including a light source for producing alignment and therapeutic light, a scanning device for deflecting the alignment and therapeutic light to produce an alignment therapeutic patterns of the alignment and therapeutic light, and an ophthalmic lens assembly for placement over a patient's eye that includes a reflective optical element for reflecting the light patterns onto the trabecular meshwork of the patient's eye. The reflective optical element can be a continuous annular mirror (e.g. smooth or with multiple facets) to image the entire trabecular meshwork, or a reflective optical element that moves in coordination with the deflection of the beam. Visualization of the alignment and therapeutic patterns of light on the eye can be implemented by reflection thereof off a visualization mirror that transmits a portion of light emanating from the trabecular meshwork.

U.S. patent application No. 20030109907, published Jun. 12, 2003 by Shadduck, discloses devices and techniques for light-mediated stimulation of trabecular meshwork in glaucoma therapy, including pigmentary glaucoma therapy. An apparatus and technique are provided for transscleral tight-mediated biostimulation of the trabecular plates of a patient's eye in a treatment for glaucoma or ocular hypertension. The apparatus After ablation of the various successive zones, the scleral flap is closed and, if necessary, sutured.

U.S. patent application No. 20050288745, published Dec. 29, 2005 by Andersen, describes a method and device for optical ophthalmic therapy, which includes treatment for different forms of glaucoma, including pigmentary glaucoma. An optical scanning system and method are provided for performing therapy on trabecular meshwork of a patient's eye, including a light source for producing alignment and therapeutic light, a scanning device for deflecting the alignment and therapeutic light to produce an alignment therapeutic patterns of the alignment and therapeutic light, and an ophthalmic lens assembly for placement over a patient's eye that includes a reflective optical element for reflecting the light patterns onto the trabecular meshwork of the patient's eye. The reflective optical element can be a continuous annular mirror (e.g. smooth or with multiple facets) to image the entire trabecular meshwork, or a reflective optical element that moves in coordination with the deflection of the beam. Visualization of the alignment and therapeutic patterns of light on the eye can be implemented by reflection thereof off a visualization mirror that transmits a portion of light emanating from the trabecular meshwork.

U.S. patent application No. 20030109907, published Jun. 12, 2003 by Shadduck, discloses devices and techniques for light-mediated stimulation of trabecular meshwork in glaucoma therapy, including pigmentary glaucoma therapy. An apparatus and technique are provided for transscleral light-mediated biostimulation of the trabecular plates of a patient's eye in a treatment for glaucoma or ocular hypertension. The apparatus includes; (i) a working end geometry for contacting the anterior surface of the sclera and cornea to insure that a laser emission reaches the trabecular meshwork from a particular location on the anterior surface of the sclera, (ii) a laser energy source providing a wavelength appropriate for absorption beneath the anterior scleral surface to the depth of the trabecular plates, and (iii) a dosimetry control system for controlling the exposure of the laser emission at the particular spatial locations. The device uses a light energy source that emits wavelengths in the near-infrared portion of the spectrum, preferably in the range of about 1.30 .mu.m to 1.40 .mu.m or from about 1.55 .mu.m to 1.85 .mu.m. The depth of absorption of such wavelength ranges will extend through most, if not all, of the thickness of the sclera (750 .mu.m to 950 .mu.m). In accordance with a proposed method of trabecular biostimulation, the targeted region is elevated in temperature to a range between about 40.degree. C. to 55.degree. C. for a period of time ranging from about 1 second to 120 seconds or more.

U.S. Pat. No. 6,306,127, issued Oct. 23, 2001 to Homer, is for a method for altering iris pigmentation in a human, thereby altering perceived iris color of a first iris from a first iris color to a second iris color. The method comprises pre-selecting one or more than one laser capable of generating one or more than one laser beam which will selectively remove iris pigment of a first pre-selected pigment color from the first iris, and applying the one or more than one laser beam to the first iris of a first iris color to remove iris pigment of the first pre-selected pigment color.

What is needed is a method and scraping tool for treating secondary pigmentary glaucoma by removing the build-up of pigment debris on the back pigment epithelium layer on the backside of the iris without harming the epithelium layer to remove the debris which blocks the flow of fluid between the iris and the lens.

### BRIEF SUMMARY OF THE INVENTION

An object of the present invention is to provide a method and scraping tool for treating secondary pigmentary glaucoma by removing the build-up of pigment debris and/or cellular debris on the back pigment epithelium layer on the backside of the iris without harming the epithelium layer to remove the debris which blocks the flow of fluid between the iris and the lens.

In brief, the present invention provides an iris scraping procedure to cure a type of secondary pigmentary glaucoma where the pigment build-up on the back pigment epithelium layer on the backside of the iris causes a blockage of fluid drainage between the iris and the lens. The method comprises partially cutting the film layer (sclera) over the iris with a separate surgical cutting tool and lift one edge of the iris and use a pointed sharp tipped double edged flat blade and handle surgical tool having two opposing dull non cutting scraping blade edges extending back from the front tip, the entire aluminum handle and aluminum blade including the tip, and scraping blade edges having a durable plastic coating to withstand operating conditions and all forms of sterilization procedures. The aluminum scraping tool is used to carefully scrape the pigment debris and dead cell tissue from the natural back pigment epithelium layer on the backside of the iris without disturbing the pigment epithelium layer, and lower the iris back down and re-attach the iris by suturing or other means.

The Iris “T” Aluminum Scraping Secondary Pigmentary Glaucoma Treatment Tool of the present invention is used to gently clean off all of the debris (build up of pigment and scarring tissue) from the back pigment epithelium layer on the backside of the iris by lifting the iris and gently scraping away the debris, without puncturing, disturbing, mutilating, or destroying the natural existence of the permanent natural back pigment epithelium layer on the backside of the iris (thin covering skin tissue) that must be left on the iris, as its natural coating. Only the debris and built up mass tissues that have accumulated and attached to the natural back pigment epithelium layer on the backside of the iris are gently scraped off leaving the natural and permanent back pigment epithelium layer on the backside of the iris. The outer face of the iris is never touched. By removing the debris, the eye fluid flows more freely between the iris and the lens to release the pressure in the eye from the build up blocking the fluid flow, thereby healing the secondary pigmentary glaucoma.

The procedure of the present invention brings forth brighter vision and clearer vision to improve eyesight and enable a person to see sharper images.

The surgical procedure method of the present invention cleans the back pigment epithelium layer on the backside of the iris to restore vision and relieve the pressure.

An advantage of the present invention is that it relieves the pressure of the fluid build-up by removing the blockage to relieve the pain of the patient and also provide brighter and clearer vision.

### BRIEF DESCRIPTION OF THE SEVERAL VIEWS OF THE DRAWINGS

These and other details of my invention will be described in connection with the accompanying drawings, which are furnished only by way of illustration and not in limitation of the invention, and in which drawings:

FIG. 1 is a partial cross-sectional view taken through the eye showing a portion of the iris and a portion of the lens with a clear passage of fluid between the iris and the lens in a healthy eye;

FIG. 2 is a partial cross-sectional view taken through the eye showing a portion of the iris and a portion of the lens with a build-up of pigment debris on the back pigment epithelium layer on the backside of the iris blocking the flow of fluid between the iris and the lens in an eye having a secondary pigmentary glaucoma condition;

FIG. 3 is an enlarged partial cross-sectional view of FIG. 2 showing the build-up of pigment debris on the back pigment epithelium layer on the backside of the iris blocking the flow of fluid between the iris and the lens in an eye having a secondary pigmentary glaucoma condition;

FIG. 4 is a partial cross-sectional view taken through the eye showing a portion of the iris and a portion of the lens with the iris lifted and an incision in the sclera, made by a separate cutting instrument, allowing the iris scraping tool of the present invention to be inserted through the incision to remove a build-up of pigment debris on the back pigment epithelium layer on the backside of the iris to free the flow of fluid between the iris and the lens;

FIG. 5 is a side elevational view of the iris scraping tool of the present invention showing the double edge dull non cutting aluminum scraping blade and aluminum handle tool coated with a chemical and heat resistant plastic;

FIG. 6 is a perspective view of the iris scraping tool of FIG. 5 showing the double edge dull non cutting aluminum scraping blade and aluminum handle tool coated with a chemical and heat resistant plastic.

### DETAILED DESCRIPTION OF THE INVENTION

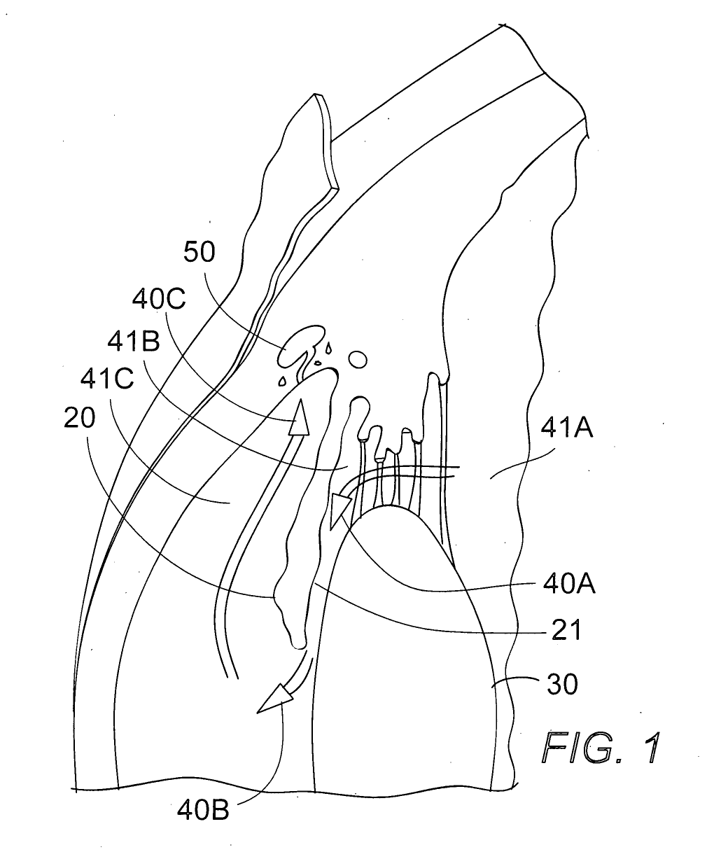

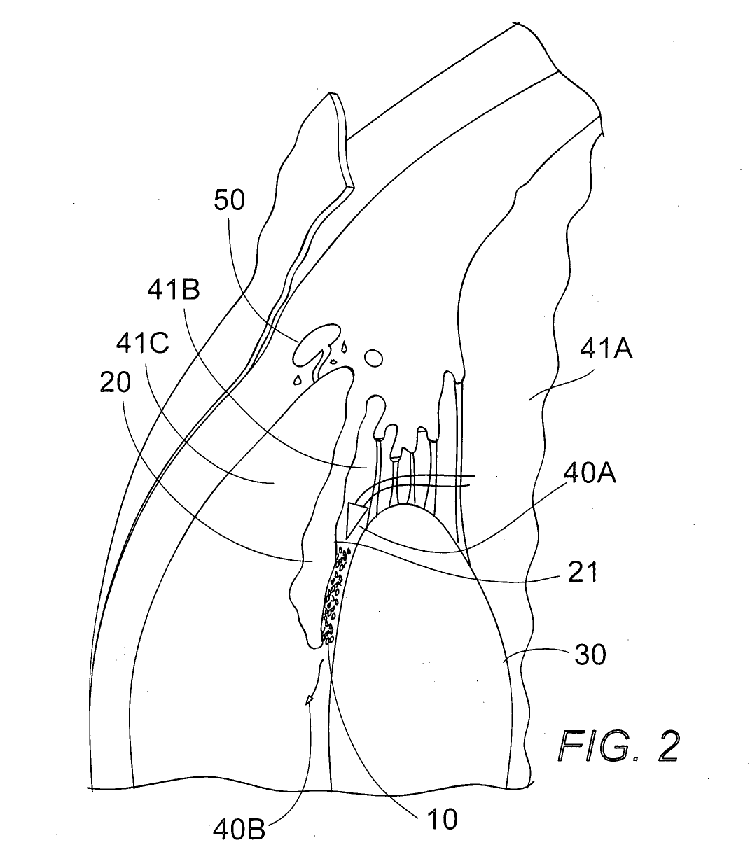

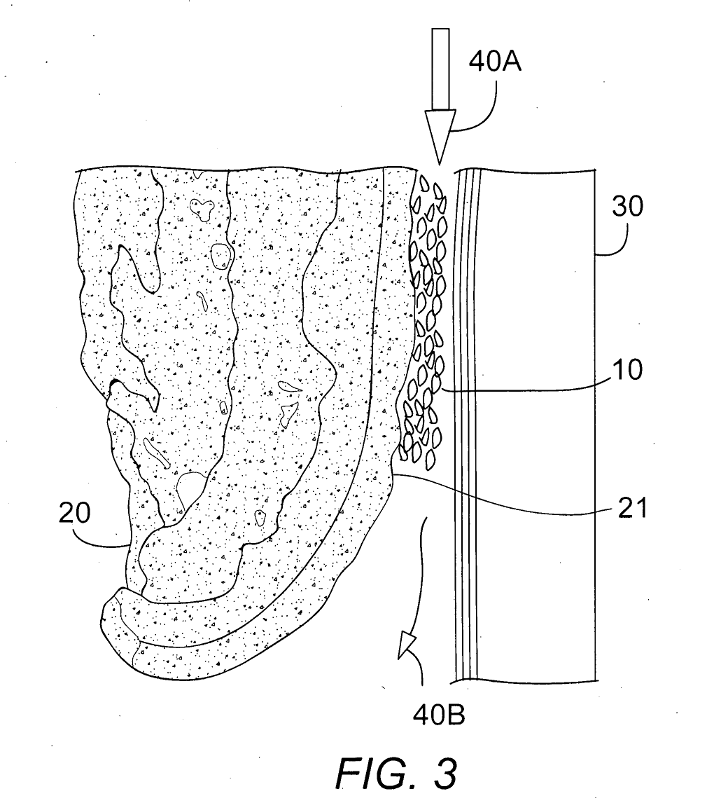

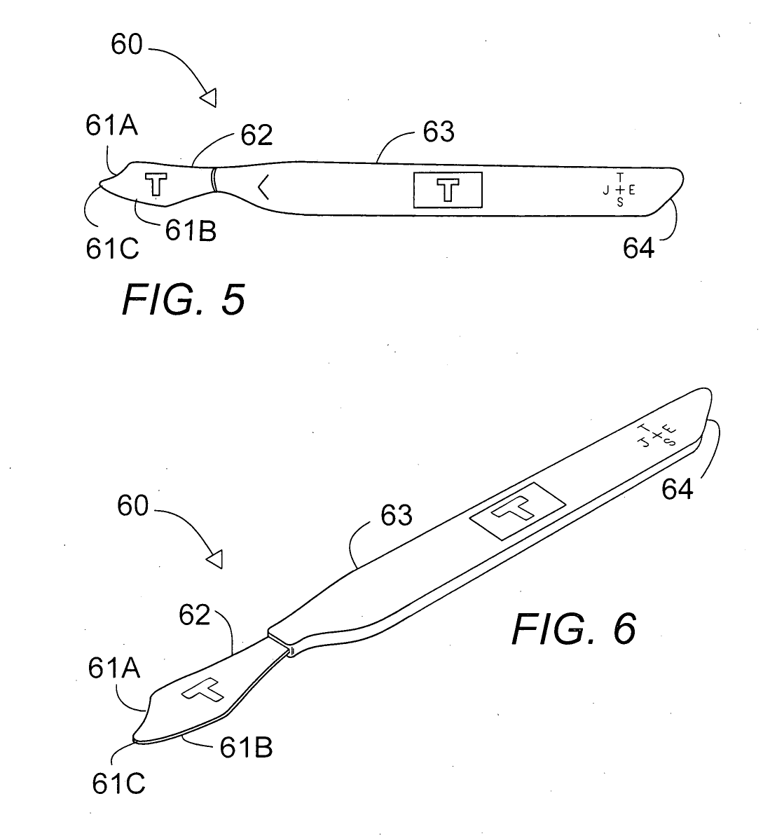

In FIGS. 1-6, an iris scraping surgical treatment method and an iris scraping tool 60 of the present invention are used for curing secondary pigmentary glaucoma by removing pigment debris 10 built up on a natural back pigment epithelium layer 21 on a backside of an iris 20 to unblock fluid flow 40A, as shown blocked in FIG. 2 and partially blocked with a reduced flow 40B in FIG. 3, between the iris 20 and the lens 30 of the eye to restore full aqueous humor fluid flow 40B, as in FIG. 1, from a vitreous humor 41A and posterior chamber 41B of the aqueous humor out between the iris 21 and the lens 30 to an anterior chamber 41C of the aqueous humor, and release the pressure buildup caused by the blocked fluid flow 40A of FIGS. 2 and 3, relieving the pressure on the eye and curing the secondary pigmentary glaucoma.

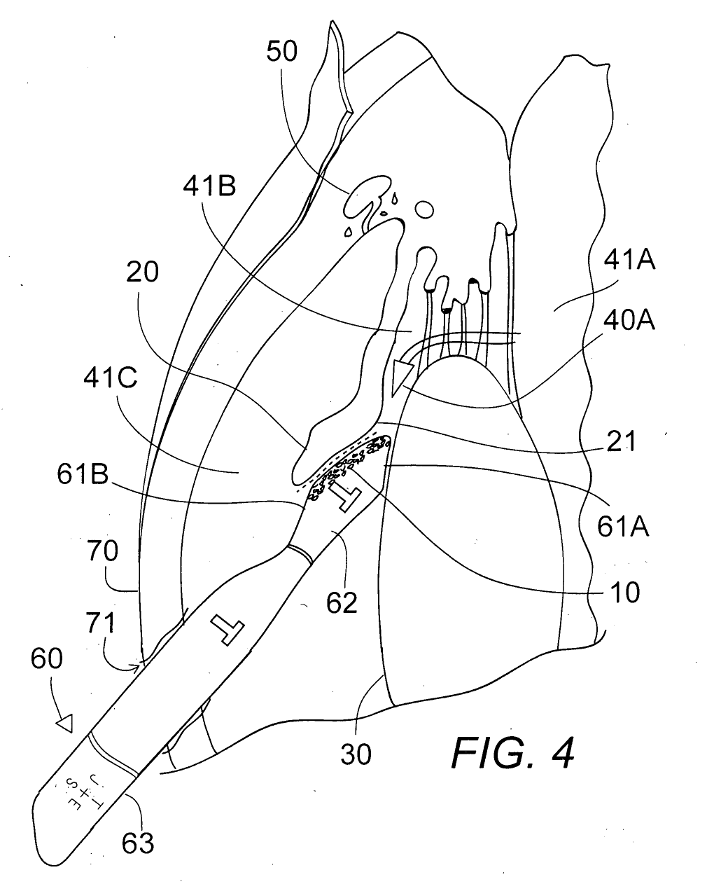

In FIG. 4, the iris scraping surgical treatment method for curing secondary pigmentary glaucoma of the present invention comprises:

a first step of making an incision 71 in an outer sclera 70 of the eye using a separate cutting tool to admit a non-cutting iris scraping tool 60 through the incision to reach an iris 20 and an adjacent lens 30 behind the outer sclera 70;

a second step of lifting the iris 20 from the lens 30 to admit a scraping head 62 on a distal end of the non-cutting iris scraping tool 60 between the iris 20 and the lens 30;

a third step of gently scraping the pigmentary debris 10 and cellular debris from a natural back pigment epithelium layer 21 on an underside of the iris 20 between the iris 20 and the lens 30 using a substantially planar scraping head 62 on the distal end of the dull non-cutting iris scraping tool 60, a convexly curved dull scraping edge 61B on one side of the scraping head structured to conform to the epithelium layer 21 on the underside of the iris lifted away from an adjacent lens 30, the convexly curved dull scraping edge 61B gently scraping away and removing excess blocking pigmentary debris 10 and cellular debris from the underside of the iris with no damage to the iris pigment epithelium layer 21; and a concave dull scraping edge 61A on an opposing side of the scraper head structured to conform to a curvature of the adjacent lens 30, the concave scraping edge 61A gently wiping and removing any pigmentary debris 10 and cellular debris from the lens 30 with no damage to the lens;

a fourth step of lowering the iris back down and suturing to close the incision in the sclera;

thereby providing an iris scraping surgical treatment method for curing secondary pigmentary glaucoma by removing the pigmentary debris 10 and cellular debris blocking aqueous humor fluid flow 40A between the iris 20 and the lens 30 and relieving the pressure build-up in the eye to cure the secondary pigmentary glaucoma.

The third step comprises using a scraping tool 62 fabricated with a flat elongated aluminum handle 63 and a heat and chemical resistant plastic coating over the handle 63 and scraping head 62 to withstand operating conditions and all forms of sterilization procedures and to provide light weight ease of handling assuring a gentler cleaning, added care, precaution, protection and security to prevent the existing natural epithelium layer 21 covering the back side of the iris 20 from being disturbed.

In FIGS. 4, 5 and 6, an iris scraping surgical treatment tool 60 for curing secondary pigmentary glaucoma caused by pressure build-up in an eye from a blockage of aqueous humor fluid flow 40A caused by excess pigmentary debris and cellular debris build-up between a natural back pigment epithelium layer 21 on a backside of an iris 20 and an adjacent lens 30, the tool structured for the removal of the debris and the restoration of the aqueous humor fluid flow 40A from a vitreous humor 41A and posterior chamber 41B of the aqueous humor out between the iris 20 and the lens 30 to an anterior chamber 41C of the aqueous humor, and the relief of the pressure and the cure of the secondary pigmentary glaucoma.

The iris scraping tool 60 comprises an elongated handle 63 having an angled back edge 64 at a proximal end and a double dull edged scraping head 62 at a distal end. The scraping head 62 comprises a substantially planar rigid surface comprising one convexly curved dull scraping edge 61B structured to conform to an underside of an iris 20 of an eye lifted away from an adjacent lens 30, as shown in FIG. 4, for the gentle scraping removal of excess blocking pigmentary debris 10 and cellular debris from the underside of the iris with no damage to the iris pigment epithelium layer 21. The scraping head 62 further comprising on an opposite edge of the head, a concave dull scraping edge 61A structured to conform to a curvature of the adjacent lens 30 of the eye for the gentle removal of any pigmentary debris 10 and cellular debris from the lens 30 with no damage to the lens 30. The scraping head 62 further comprising a small rounded dull distal non-cutting end 61C between the two curved dull scraping edges so that the dull end 61C cannot puncture any parts of the eye while in use.

The scraping tool 60 is structured for use in the method for curing secondary pigmentary glaucoma, wherein a first step of the method forms an incision 71 in an outer sclera 70 of the eye using a separate cutting tool to admit the iris scraping tool 60 through the incision 71 to reach an iris 20 and an adjacent lens 30 behind the outer sclera 70. A second step of the method produces a lifting of the iris 20 away from the lens 30 to admit the scraping head 62 of the iris scraping tool 60 between the iris 20 and the lens 30, as shown in FIG. 4. A third step of the method utilizes the scraping edges 61A and 61B of the scraping head 62 for the removal of the pigmentary debris 10 and cellular debris from the natural back pigment epithelium layer 21 on a backside or underside of the iris 20 between the iris 20 and the lens 30.

Thereby providing an iris scraping surgical treatment tool 60 for curing secondary pigmentary glaucoma caused by pressure build-up in an eye from blocked aqueous humor fluid flow 40A caused by excess pigmentary debris 10 and cellular debris build-up between a natural back pigment epithelium layer 21 on a backside of an iris 20 and an adjacent lens 30. The iris scraping tool 60 is structured for the removal of the debris and the restoration of the aqueous humor fluid flow 40A from the vitreous humor 41A and posterior chamber 41 B of the aqueous humor out between the iris 20 and the lens 30 to an anterior chamber 41C of the aqueous humor, for the relief of fluid build-up pressure and the cure of the secondary pigmentary glaucoma.

The iris scraping tool 60 is preferably fabricated with a flat elongated aluminum handle 63 and a heat and chemical resistant plastic coating over the handle 63 and aluminum scraping head 62 to withstand operating conditions and all forms of sterilization procedures and to provide light weight ease of handling assuring a gentler cleaning, added care, precaution, protection and security to prevent the existing natural epithelium layer 21 covering the back side of the iris 20 from being disturbed, although both may be stainless steel if necessary. The length of the iris scraping surgical tool is preferably 5.5620 inches long. The handle is preferably 4.3699 inches long and 0.1275 inches in thickness. The length of the scraping blade is preferably 0.4400 inches and the length of the blade double edges is preferably 1.6321 inches.

CAUTION: DO NOT PUNCTURE, DAMAGE, DESTROY, OR REMOVE THE EXISTING FILM THAT IS PERMANENTLY ATTACHED TO THE IRIS.

NOTE: ONLY ‘ALL’ DEBRIS AND DEAD CELL TISSUES CAN BE REMOVED.

It is understood that the preceding description is given merely by way of illustration and not in limitation of the invention and that various modifications may be made thereto without departing from the spirit of the invention as claimed.

What is claimed is:

1. An iris scraping surgical treatment method for curing secondary pigmentary glaucoma caused by pressure build-up in an eye from blocked aqueous humor fluid flow caused by excess pigmentary debris and cellular debris build-up between a natural back pigment epithelium layer on a backside of an iris and an adjacent lens, the method removing the debris and restoring the aqueous humor fluid flow from a vitreous humor and posterior chamber of the aqueous humor out between the iris and the lens to an anterior chamber of the aqueous humor, relieving the pressure and curing the secondary pigmentary glaucoma, the method comprising:
a first step of making an incision in an outer sclera using a separate cutting tool to admit a non-cutting iris scraping tool through the incision to reach an iris and an adjacent lens behind the outer sclera;
a second step of lifting the iris from the lens to admit a scraping head on a distal end of the non-cutting iris scraping tool between the iris and the lens;
a third step of gently scraping the pigmentary debris and cellular debris from a natural back pigment epithelium layer on an underside of the iris between the iris and the lens using a substantially planar scraping head on the distal end of the dull non-cutting iris scraping tool, a convexly curved dull scraping edge on one side of the scraping head structured to conform to the epithelium layer on the underside of the iris lifted away from an adjacent lens, the convexly curved dull scraping edge gently scraping away and removing excess blocking pigmentary debris and cellular debris from the underside of the iris with no damage to the iris pigment epithelium layer; and a concave dull scraping edge on an opposing side of the scraper head structured to conform to a curvature of the adjacent lens, the concave scraping edge gently wiping and removing any pigmentary debris and cellular debris from the lens with no damage to the lens;
a fourth step of lowering the iris back down and suturing to close the incision in the sclera;
thereby providing an iris scraping surgical treatment method for curing secondary pigmentary glaucoma by removing the pigmentary debris and cellular debris blocking aqueous humor fluid flow between the iris and the lens and relieving the pressure build-up in the eye to cure the secondary pigmentary glaucoma.

2. The method of claim 1 wherein the third step comprises using a scraping tool fabricated with a flat elongated aluminum handle and a heat and chemical resistant plastic coating over the handle and scraping head to withstand operating conditions and all forms of sterilization procedures and to provide light weight ease of handling assuring a gentler cleaning, added care, precaution, protection and security to prevent the existing natural epithelium layer covering the back side of the iris from being disturbed.

3. An iris scraping surgical treatment tool for curing secondary pigmentary glaucoma caused by pressure build-up in an eye from a blockage of aqueous humor fluid flow caused by excess pigmentary debris and cellular debris build-up between a natural back pigment epithelium layer on a backside of an iris and an adjacent lens, the tool structured for the removal of the debris and the restoration of the aqueous humor fluid flow from a vitreous humor and posterior chamber of the aqueous humor out between the iris and the lens to an anterior chamber of the aqueous humor, and the relief of the pressure and the cure of the secondary pigmentary glaucoma, the tool comprising:
an iris scraping tool having a handle at a proximal end and a double dull edged scraping head at a distal end, the scraping head comprising a substantially planar rigid surface comprising one convexly curved dull scraping edge structured to conform to an underside of an iris of an eye lifted away from an adjacent lens for the gentle scraping removal of excess blocking pigmentary debris and cellular debris from the underside of the iris with no damage to the iris pigment epithelium layer; the scraping head further comprising on an opposite edge of the head, a concave dull scraping edge structured to conform to a curvature of the adjacent lens of the eye for the gentle removal of any pigmentary debris and cellular debris from the lens with no damage to the lens; and the scraping head further comprising a small rounded dull distal non-cutting end between the two curved dull scraping edges;
the scraping tool structured for use in a method for curing secondary pigmentary glaucoma, wherein a first step of the method forms an incision in an outer sclera of the eye using a separate cutting tool to admit the iris scraping tool through the incision to reach an iris and an adjacent lens behind the outer sclera; and a second step of the method produces a lifting of the iris away from the lens to admit the scraping head of the iris scraping tool between the iris and the lens; and a third step of the method utilizes the scraping edges of the scraping head for the removal of the pigmentary debris and cellular debris from a natural back pigment epithelium layer on a backside of the iris between the iris and the lens;
thereby providing an iris scraping surgical treatment tool for curing secondary pigmentary glaucoma caused by pressure build-up in an eye from blocked aqueous humor fluid flow caused by excess pigmentary debris and cellular debris build-up between a natural back pigment epithelium layer on a backside of an iris and an adjacent lens, the tool structured for the removal of the debris-and the restoration of the aqueous humor fluid flow from a vitreous humor and posterior chamber of the aqueous humor out between the iris and the lens to an anterior chamber of the aqueous humor, for the relief of fluid build-up pressure and the cure of the secondary pigmentary glaucoma.

4. The iris scraping tool of claim 3 wherein the iris scraping tool is fabricated with a flat elongated aluminum handle and a heat and chemical resistant plastic coating over the handle and scraping head to withstand operating conditions and all forms of sterilization procedures and to provide light weight ease of handling assuring a gentler cleaning, added care, precaution, protection and security to prevent the existing natural epithelium layer covering the back side of the iris from being disturbed.
